(19) United States
(12) Patent Application Publication
(10) Pub. No.: US 2024/0316039 A1
(43) Pub. Date: Sep. 26, 2024

(54) METHOD OF REDUCING ELEVATED INTRAOCULAR PRESSURE

(71) Applicant: SUN PHARMA ADVANCED RESEARCH COMPANY LIMITED, Mumbai, Maharashtra (IN)

(72) Inventors: Ajay Jaysingh KHOPADE, Vadodara, Gujarat (IN); Arindam HALDER, Vadodara, Gujarat (IN)

(21) Appl. No.: 18/259,982

(22) PCT Filed: Jan. 13, 2022
(86) PCT No.: PCT/IB2022/050259

§ 371 Date: Jun. 29, 2023

(30) Foreign Application Priority Data
Jan. 13, 2021 (IN) 202121001732

(51) Int. Cl. A61K 31/498; A61K 9/00; A61K 9/10; A61P 27/06

(52) U.S. Cl. CPC A61K 31/498; A61K 9/0048; A61K 9/10; A61P 27/06

(57) ABSTRACT

The present invention relates to a method of reducing elevated intraocular pressure in humans with open angle glaucoma or ocular hypertension, comprising administering brimonidine or its pharmaceutically acceptable salt. The invention also relates to a pharmaceutical composition suitable for ophthalmic use comprising brimonidine or its pharmaceutically acceptable salt.

### FIELD OF THE INVENTION

The present invention relates to a method of reducing elevated intraocular pressure in humans with open angle glaucoma or ocular hypertension, comprising administering brimonidine or a pharmaceutically acceptable salt thereof. The present invention further relates to an aqueous suspension comprising brimonidine and its pharmaceutical use.

### BACKGROUND OF THE INVENTION

Glaucoma is a leading cause of irreversible blindness worldwide. It is commonly associated with elevated intraocular pressure, which leads to damages to retinal ganglion cells, and can progress to blindness if untreated. Intraocular pressure is the measure of the dynamic balance between the secretion (inflow) and drainage (outflow) of the aqueous humour, which is a fluid continuously secreted by the ciliary epithelium of the eye. Once secreted, the aqueous humour enters the eye from the anterior chamber through the pupil and exits mainly through the trabecular meshwork, or to a lesser extent, through the uveoscleral outflow pathway. The trabecular meshwork drains the aqueous humour via the Schlemm's canal into the scleral plexuses and general blood circulation. Elevated intraocular pressure results from insufficient drainage of aqueous humour from the eye, and is a major risk factor for glaucoma.

There are two major types of glaucoma: open-angle glaucoma and closed-angle glaucoma. Open-angle glaucoma is caused by reduced drainage of aqueous humour due to blockages in the trabecular meshwork, resulting in elevated intraocular pressure. Closed-angle glaucoma is characterized by contact between the iris and the trabecular meshwork, which obstructs the outflow of aqueous humour, leading to an increase in intraocular pressure. In about 50% of closed-angle glaucoma cases, prolonged contact between the iris and the trabecular meshwork results in the formation of synechia, which causes permanent obstruction of aqueous outflow.

“Ocular hypertension” refers to an elevated intraocular pressure above the normal level, which is not accompanied by any functional impairment of vision, but, after a long period of time, may develop glaucoma. The medical treatment of ocular hypertension or glaucoma is directed to the reduction of the elevated intraocular pressure down to the normal IOP that induces no functional impairment, and also directed to the maintenance of the normal IOP.

Ophthalmic drug delivery is one of the most challenging endeavors facing the pharmaceutical scientist. The eye is a unique organ, both anatomically and physiologically, containing several widely varied structures with independent physiological functions. The complexity of the eye provides unique challenges to drug delivery strategies. Typically, the ocular bioavailability of drugs applied topically as eye-drops is very poor. The absorption of drugs in the eye is severely limited by some protective mechanisms that ensure the proper functioning of the eye, and by other concomitant factors, for example: nasolacrimal drainage of the instilled solutions; lacrimation and tear turnover; low corneal contact time; metabolism; tear evaporation; non-productive absorption/adsorption; limited corneal area and poor corneal permeability; and binding by the lacrimal proteins. These factors have a huge effect on ocular drug absorption and disposition and lead to low ocular drug bioavailability. Thus, developing an ocular drug delivery system that provides optimum drug bioavailability is a challenge.

Brimonidine tartrate is an alpha-2-adrenergic agonist that reduces the elevated intraocular pressure (IOP) of the eye that is associated with glaucoma. The topical use of brimonidine to lower intraocular pressure in patients with glaucoma or ocular hypertension is known.

Brimonidine typically causes moderate peak IOP reduction of about 20-25% in ocular hypertensive eyes and 6-18% in normotensive eyes (less than 21 mm Hg). Its peak effect is within 2 hours of instillation, its duration of effect is typically less than 12 hours, and its moderate efficacy usually requires dosing of 2-3 times a day.

The first ophthalmic brimonidine product in the U.S. was approved by the FDA in 1996. That product, sold under the trade name Alphagan®, contained brimonidine in the form of brimonidine tartrate at a concentration of 0.2%. In 2001, a second ophthalmic brimonidine product was approved by the U.S. FDA. This product, sold under the trade name Alphagan® P, it contained brimonidine tartrate at two brimonidine concentrations, 0.15% and 0.1%, each of which is lower than the 0.2% brimonidine concentration in Alphagan®.

It is one objective of this invention to provide a composition for treating ocular hypertension or glaucoma, having an excellent ocular hypotensive effect (i.e., intraocular pressure-reducing effect) and no significant side effects, in particular, a minimal eye irritating effect.

It is another objective of this invention to provide an efficient and stable ophthalmic drug delivery system which upon ocular administration, leads to an increase in ocular bioavailability of the drug, prolongs the ophthalmic action of the drug, is more convenient to use, and minimizes the irritation or other discomfort associated with ocular application.

The present invention fulfills this need and provides an ophthalmic composition in the form of a novel aqueous suspension comprising brimonidine or its pharmaceutically acceptable salt that can be instilled into the eye once-a-day which is effective in reducing elevated intraocular pressure in humans with open angle glaucoma or ocular hypertension.

The novel compositions of present invention provide equivalent efficacy to that of Alphagan P®, but at a reduced frequency of administration, that is once daily instillation as compared to thrice daily instillation, as prescribed for Alphagan P®

### SUMMARY OF THE INVENTION

A first aspect of the present invention relates to an aqueous suspension of: (a) reversible clusters of brimonidine loaded nano-resin particles and (b) a suspending agent, wherein said brimonidine loaded nano-resin particles have a particle size distribution wherein the D90 value is between 70 to 900 nm and D50 value is between 50-700 nm, and wherein said suspension is for use in the treatment of elevated intraocular pressure in human patients suffering from open angle glaucoma or ocular hypertension.

The present invention further relates to a method for the treatment of elevated intraocular pressure in human patients suffering from open angle glaucoma or ocular hypertension, comprising administering to a patient in need thereof the aqueous suspension as referred to above.

In a further aspect, the present invention relates to a method of reducing elevated intraocular pressure in human patients with open angle glaucoma or ocular hypertension, comprising administering brimonidine or its pharmaceutically acceptable salt, or the aqueous suspension as referred to above, wherein said method lowers the intraocular pressure in said patients by at least 2-6 mmHg; and wherein said method is effective in reducing the intraocular pressure over a time period of about 7 to 15 weeks

Another aspect of the present invention relates to a method of reducing elevated intraocular pressure in human patients with open angle glaucoma or ocular hypertension, comprising administering brimonidine or its pharmaceutically acceptable salt, or an aqueous suspension as referred to above, wherein after administration patients achieved >20% intraocular pressure reduction from baseline which is sustained for at least 12 weeks.

### DETAILED DESCRIPTION OF THE INVENTION

The present invention relates to an aqueous suspension of: (a) reversible clusters of brimonidine loaded nano-resin particles and (b) a suspending agent, wherein said brimonidine loaded nano-resin particles have a particle size distribution wherein the D₉₀ value is between 70 to 900 nm and D₅₀ value is between 50-700 nm, and wherein said suspension is for use in the treatment of elevated intraocular pressure in human patients suffering from open angle glaucoma or ocular hypertension. Said aqueous suspension has surprisingly been able to increase bioavailability of brimonidine along with prolonged ophthalmic action.

Without wishing to be bound by any theory, it is believed that the reversible clusters of the brimonidine nano-resin particles upon instillation into the eye, decluster under shear due to eye blinking. The declustered individual drug-loaded nano-resin particles spread on the surface of the cornea and subsequently releases the drug through ion exchange phenomenon under the effect of ions present in the ocular tissue. There occurs a decrease in naso-lacrymal drainage of the drug. The overall phenomenon causes increased bioavailability of the drug along with prolonged ophthalmic action.

Preferably, the D₉₀ value of the nano-resin particles ranges between 200 and 700 nm. The present inventors have found that said aqueous suspension is particularly efficacious when said particles are within said range.

Furthermore, the D₅₀ of the nano-resin particles preferably ranges between 50 to 700 nm, more preferably between 100 to 500 nm, alternatively between 150 to 350 nm or between 200 to 300 nm.

Moreover, the reversible clusters of brimonidine loaded nano-resin particles of the aqueous suspension according to the present invention may comprise brimonidine as a pharmaceutically acceptable salt or solvate. A particularly preferred salt is brimonidine tartrate.

As used herein, the term “IOP” wherever it appears is an abbreviation for “intraocular pressure”. Increased IOP of greater than 21 mm Hg has traditionally been suspected to cause glaucoma (7); the higher the IOP, the greater the likelihood of optic nerve damage and visual field loss. With IOP>30 mmHg, the potential risk for vision loss is 40 times greater compared to an IOP of 15 mm Hg.

The terms “reducing” and “lowering” refer to prophylactic use to reduce the likelihood of a disease, disorder, or condition to which such term applies, or one or more symptoms of such disease, disorder, or condition.

The term “Reversible Clusters” of brimonidine loaded nano-resin particles, according to the present invention, means that individual brimonidine loaded nanoresin particles form aggregates or agglomerates, which preferably have a mean size of about 2 micrometers or greater which upon application of shear deagglomerate or decluster into individual brimonidine loaded nano-resin particles.

Qualitatively, the declustering can be observed by microscopy (Morphology G3S-ID™ Instrument, Make: Malvern®) by observing sheared (by smearing) and unsheared samples onto a glass slide.

As referred to further herein, the D₅₀ of the clusters is obtained from the particle size distribution obtained before the application of shear. The particle size distribution may be determined in a suitable particle size analyser such as the Malvern Mastersizer 2000®. The particle size analyser's sonication means are not used but the sample is only subjected to stirring by a mechanical stirrer.

The suspension of clusters is placed in a sonication bath and is subjected to shear using a sonication frequency of about 33±3 kHz for 5 seconds, and a sample is withdrawn to measure the particle size distribution. Following intervals of 1 minute each, the process is repeated 5 times. (See Example 7)

Nanoresin particles or drug-loaded nanoresin particles according to the present invention mean individual ion exchange resin particles and not clusters or agglomerates of the individual particles, which individual particles have a particle size distribution characterized in that the D₉₀ value is in the range of about 70 nanometer to 900 nanometers, preferably 200 to 700 nanometers. The particle size distribution of the nanoresin particles may be considered as the particle size distribution obtained after the suspension has been subjected to 5 pulses of a frequency of 33±3 KHz with intervals of 1 minute each, as described above.

The individual brimonidine loaded nano-resin particles have a particle size distribution value of D₉₀ of 70 nm to 900 nm, preferably 200 nm to 700 nm. Further, such drug-loaded nanoresin particles may have a mean particle size (D₅₀) of less than 700 nm. Preferably, the D₅₀ of the nano-resin particles is 50 to 700 nm, preferably 100 to 500 nm, alternatively 150 to 350 nm or 200 to 300 nm. The particle size distribution of the nanoresin may be further characterized in that the D₁₀ is less than 300 nm, preferably 10 nm to 250 nm.

The reversible clusters have a particle size distribution such that the mean size is preferably at least 2 micrometer (μm or microns). In further preferred embodiments, the particle size distribution of said clusters is such that the D₉₀ thereof is less than 80 micrometers, preferably less than 50 micrometers, most preferably less than 30 micrometers. The particle size distribution may be further characterized in that the D₅₀ is less than 40 micrometers, preferably less than 20 micrometers, and more preferably less than 10 micrometers. As said, the reversible clusters, upon application of shear deagglomerate or deaggregate to form brimonidine loaded nanoresin particles.

The present invention provides a method of reducing elevated intraocular pressure in humans comprising administering an aqueous suspension comprising: (a) reversible clusters of brimonidine loaded nano-resin particles, said clusters having a D₅₀ value of at least 2 micrometers and said brimonidine loaded nano-resin particles having a particle size distribution characterized in that the D₉₀ value is 70 nm to 900 nm, and D₅₀ value is 50 nm to 700 nm, and (b) a suspending agent, and (c) pharmaceutically acceptable excipients.

The present inventors have surprisingly found that it is of importance to monitor the patient that is administered said aqueous suspension for specific side effects. For instance, it is important to monitor if the patients that have been administered the suspension according to the present invention do not suffer from, for example, nervous system disorders, ocular hyperemia, somnolence or gastrointestinal disorders. In view of this, it is important to provide, in addition to administration of the aqueous suspension according to the present invention, patient monitoring. Said patient monitoring comprises an ophthalmic examination for the appearance of one or more of potential side effects. In order to avoid permanent damage to the eye it is of further importance that said examination be performed within a short period of time after commencing the treatment with the aqueous suspension, such as within 16 weeks, optionally within 12 weeks.

Hence, optionally the above mentioned treatment with the aqueous suspension according to the present invention further comprises the steps of:
(a) identifying the eye disease history of a patient to be treated before commencing the treatment with the aqueous suspension;
(b) having ophthalmic examinations performed within at least 16 weeks, preferably within at least 12 weeks, after commencing the treatment with the aqueous suspension, preferably by an ophthalmologist, said examinations comprising checking and/or monitoring the appearance of somnolence, ocular hyperemia, nervous system disorders or oral dryness;
(c) having optional additional ophthalmologic examinations performed based on patient symptoms at intervals determined by the ophthalmologist.

In further embodiments of the aqueous suspension and its use according to the present invention, brimonidine or its pharmaceutically acceptable salt is present at a concentration of 0.05% to 0.5% weight by volume. In a preferred embodiment, the pharmaceutically acceptable salt is brimonidine tartrate. Most preferably, said suspension comprises about 0.35% weight by volume of brimonidine tartrate. In a further embodiment, the suspension comprises about 0.2% to about 0.5% weight by volume of brimonidine or a pharmaceutically acceptable salt thereof, in particular brimonidine tartrate.

In one embodiment, brimonidine tartrate is present in said suspension at a concentration of 0.35% weight by volume. In another embodiment, the aqueous suspension of the present invention contains brimonidine tartrate at a concentration of 0.15% weight by volume.

In a further embodiment, the resin is polystyrene divinyl benzene sulfonate. In another embodiment, the suspending agent is a mixture of carbopol, polyvinylpyrrolidone and hydroxypropylmethyl cellulose. In yet another embodiment, the resin is polystyrene divinyl benzene sulfonate, and the suspending agent is a mixture of carbopol, polyvinylpyrrolidone and hydroxypropylmethyl cellulose. The weight ratio between the nano-resin particles and brimonidine is about 1:1; and the pH is about 7 to 8.

In another embodiment, the resin is a cation exchange resin. The matrix in cationic exchangers carries ionic groups such as sulfonic, carboxylate and phosphate groups. The matrix in anionic exchangers carries primary, secondary, tertiary or quaternary ammonium groups. The resin matrix determines its physical properties, its behavior towards biological substances, and to a certain extent, its capacity. Since brimonidine has a positive charge, it can bind with cation exchange resins. Sulfonic acid exchangers are the most common cation exchange resins used for drug resonate aqueous suspensions of the present invention. In general, they are cross-linked polystyrenes with sulfonic acid groups which have been introduced after polymerization by treatment with sulfuric acid or chlorosulfonic acid. Suitable cation exchange resins that may be used in the present invention include, but are not limited to, sodium polystyrene divinyl benzene sulphonate, such as that marketed by Rohm and Haas, under the trade name Amberlite™ IRP69; polacrilex resin which is derived from a porous copolymer of methacrylic acid and divinylbenzene, such as that marketed by Rohm and Haas, under the trade name Amberlite™ IRP64; polacrilin potassium, which is a potassium salt of a cross linked polymer derived from methacrylic acid and divinylbenzene, such as that marketed by Rohm and Haas, under the trade name Amberlite™ IRP88. The resins marketed by the company Ion Exchange India Ltd., under the tradenames such as INDION™234; INDION™264; INDION™ 204; INDION™ 214 may also be used. In one embodiment, the preferred resin used in the present invention is Amberlite IRP69 which is derived from a sulfonated copolymer of styrene and divinyl benzene. Amberlite IRP69 is a pharmaceutical grade strong cation exchange resin and structurally a polystyrene sulfonic acid resin cross-linked with divinyl benzene, i.e. polystyrene divinyl benzene sulfonate. Amberlite IRP69 resin is available commercially from Rhom & Haas Company. The mobile or exchangeable cation in the resin is sodium, which can be exchanged for, or replaced by, cationic (basic) species. In embodiments of the present invention, the positively charged cationic drug is bound to the negatively charged sulfonic acid groups of the Amberlite resin.

The amount of cation exchange resin present in the aqueous suspension of the present invention may range from about 0.05% to 5.0% weight by volume of the suspension. The weight ratio of resin to brimonidine may range from 0.1:1 to 1:0.1, more preferably from 0.3:1 to 1:0.3. In one preferred embodiment, the weight ratio between the nano-resin particles and brimonidine is about 1:1. Nanoresin particles used according to the present invention may have a particle size distribution characterized in that the D₉₀ value is 70 nm to 900 nm, preferably 200 nm to 700 nm.

The aqueous suspension according to the present invention may contain one or more suspending agents. The suspending agents are used to increase the viscosity of the suspension, to cause clustering of the nanoresin particles into micron size clusters, and to prevent the caking of the suspension. Suitable suspending agents are selected from an anionic polymer, a non-ionic polymer or mixtures thereof. The anionic polymers may be selected from the group consisting of polymers of acrylic acid like carboxyvinyl polymer or carbomer, also known as carbopols. Various grades of carbomers including Carbopol 934P, 974, 1342 and the like may be used in the present invention. The polymers of acrylic acid may be present in the aqueous suspension of the present invention in an amount ranging from about 0.01% to 0.5% weight by volume of the suspension. Other anionic polymers that can be used include, but are not limited to, sodium hyaluronate; sodium carboxymethylcellulose; guargum; chondroitin sulphate; and sodium alginate. Particularly, the preferred anionic polymers that may be used include Carbopol 974P. This anionic polymer is most preferably used in an amount of 0.1% w/v of the suspension. The non-ionic polymers that can be used according to the present invention may be selected from the group consisting of non-ionic polymers such as polyvinyl pyrrolidone, soluplus-a polyvinyl caprolactam-polyvinyl acetate-PEG graft co-polymer, poloxamers, polyvinyl alcohol, polypropylene glycol, cellulose derivatives like hydroxyethylcellulose, hydroxypropyl cellulose, hydroxypropyl methylcellulose, methyl cellulose, ethyl cellulose and the like. The non-ionic polymers may be present in the aqueous suspension of the present invention in an amount ranging from about 0.1% to about 5.0% weight by volume of the suspension. The preferred non-ionic polymers that may be used include hydroxypropyl methylcellulose and polyvinylpyrrolidone. Various pharmaceutically acceptable grades of hydroxypropyl methylcellulose (also known as hypromellose or HPMC or Methocel) and polyvinylpyrrolidine (also known as povidone or PVP or plasdone) may be used. The preferred grades of polyvinylpyrrolidine which can be used in the suspensions of the present invention include PVP K-30, PVP K-25, PVP K-50; PVP K-60 and PVP K-90. It may be present in the aqueous suspension in an amount ranging from about 0.5% to about 3.0% weight by volume of the suspension. The most preferred grade used in the aqueous suspension of the present invention is PVP K-90, whose 10% w/v aqueous solution has a dynamic viscosity in the range of about 300.0 cps to about 700.0 cps at 20° C., and has an approximate molecular weight of about 1,000,000 to 1,500,000. In a preferred embodiment, polyvinylpyrrolidine PVP K-90 is used in an amount of 1.2% w/v of the suspension. The preferred grades of hydroxypropylmethylcellulose which may be selected to be used in the aqueous suspensions of the present invention include, but are not limited to, METHOCEL E, (USP grade 2910/HYPROMELLOSE 2910); METHOCEL F. (USP grade 2906/HYPROMELLOSE 2906); METHOCEL A15 (Premium LV); METHOCEL A4C (Premium); METHOCEL A15C (Premium); METHOCEL A4M (Premium), HPMC USP Grade 1828 and the like. Said materials may be present in the aqueous suspension of the present invention in an amount ranging from about 0.5% to about 3.0% weight by volume of the suspension. In a most preferred embodiment, the aqueous suspension comprises Hypromellose 2910 in an amount of 0.3% w/v. As auxiliary to the suspending agents, the flocculation of nanoresin particles may also be assisted by electrolytes.

The aqueous suspensions according to the present invention may have a viscosity ranging from about 2 cps to 4000 cps, preferably about 5 cps to 400 cps. The viscosity of the aqueous suspensions may be measured by known techniques and instruments such as by Brookfield Viscometer™, under standard conditions. It is to be noted that aqueous suspensions according to one embodiment of the present invention maintain their viscosity upon instillation into the eye, i.e. the viscosity does not change substantially upon coming in contact with the eye fluid that contains various ions such as sodium, potassium, calcium, magnesium, zinc, chloride, and bicarbonate.

The aqueous suspensions according to the present invention may additionally comprise other pharmaceutically acceptable excipients such as chelating agents, preservatives and adjuvants for preservatives. The aqueous suspension may further include one or more osmotic agents/tonicity adjusting agents, one or more pharmaceutically acceptable buffering agents and/or pH-adjusting agents. These excipients may be dissolved or dispersed in a pharmaceutically acceptable aqueous vehicle such as water for injection.

In order to achieve, and subsequently maintain, an optimum pH suitable for ophthalmic preparations, the aqueous suspensions of the present invention may contain a pH adjusting agent and/or a buffering agent in suitable amounts. The preferred range of pH for the aqueous suspension is about 5.0 to about 8.0, preferably 7.0 to 8.0. The most preferred pH is about 7.4. The aqueous suspensions of the present invention comprise a pharmaceutically acceptable pH adjusting agent that may be selected from the group comprising tromethamine, acetic acid or salts thereof, boric acid or salts thereof, phosphoric acid or salts thereof, citric acid or salts thereof, tartaric acid or salts thereof, sodium hydroxide, potassium hydroxide, sodium carbonate, sodium hydrogen carbonate, trometamol, and the like and mixtures thereof. Particularly, preferred pH adjusting agents that may be used in the aqueous suspensions of the present invention include tromethamine, acetic acid, hydrochloric acid, sodium carbonate and sodium hydroxide, most preferably tromethamine.

The aqueous suspensions of the present invention may be isotonic with respect to the ophthalmic fluids present in the human eye and are characterized by osmolarities of 250-375 mOsm/kg. Osmolality is adjusted by addition of an osmotic/tonicity adjusting agent. Osmotic agents that may be used in the suspension of the present invention to make it isotonic with respect to the ophthalmic fluids present in the human eye, are selected from the group comprising sodium chloride, potassium chloride, calcium chloride, sodium bromide, mannitol, glycerol, sorbitol, propylene glycol, dextrose, sucrose, and the like, and mixtures thereof. These are used in suitable amounts to maintain the desired osmolarity. In preferred embodiments of the present invention, a non-ionic osmotic agent such as mannitol is used as the osmotic agent. Mannitol may be present in the suspension of the present invention in an amount ranging from about 2.0% to about 6.0% w/v, preferably from about 3.0% to about 5.0% w/v and most preferably about 4.5% w/v.

Further, the aqueous suspensions of the present invention may comprise preservatives in antimicrobial effective amounts. The preservative that may be used in the aqueous suspensions of the present invention may be selected from, but not limited to: quaternary ammonium compounds such as benzalkonium chloride (BKC), benzododecinium bromide, cetrimonium chloride, polixetonium and benzethonium chloride; organic mercurials such as phenylmercuric acetate, phenylmercuric nitrate and thimerosal; parabens such as methyl and propyl paraben; ethyl paraoxybenzoate or butyl paraoxybenzoate; acids and their pharmaceutically acceptable salts such as sorbic acid, potassium sorbate, ascorbic acid, boric acid, borax, salicylic acid; substituted alcohols and phenols such as chlorobutanol, benzyl alcohol, phenyl ethanol; amides such as acetamide; other preservatives like Polyquad®, polyhexamethylene biguanide, sodium perborate, aminomethyl propanol, chlorhexidine acetate, a self preserved system containing ionic preservatives like combination of zinc and borate; and the like, and combinations thereof. Preferably, the ophthalmic aqueous suspensions of the present invention comprise a ‘quaternary ammonium compound’ as a preservative, particularly benzalkonium chloride. Benzalkonium chloride is characterized as a mixture of alkyldimethylbenzylammonium chlorides. It may be employed in the aqueous suspension of the present invention in a concentration of about 0.005 to about 0.05% w/v, preferably 0.02% w/v. The aqueous suspensions may further comprise an adjuvant to a preservative in suitable amounts, such as N-lauroyl sarcosine sodium. A suitable chelating agent that may be used is edetate disodium.

The aqueous suspensions of the present invention remain physically and chemically stable during the shelf life of the product. For instance, in case of aqueous suspensions containing the drug brimonidine, there occurs no significant change in assay of brimonidine after long term storage of the suspension. The assay of the drug remained within specified limits and no degradation products or impurities were observed upon storage. Also, there was no significant increase in related substances.

It was found from a tissue distribution study that the method according to the present invention is capable of delivering a drug to the posterior segment of the eye. The ability of the present invention to deliver the drug in the posterior segment of the eye makes it suitable for treating diseases of the posterior segment, which are difficult to treat. For instance in case of brimonidine, delivery to the posterior segment, helps in preventing degeneration of neurons and imparting neuroprotection action. This was a surprising finding because the method provided not only increased bioavailability of drugs along with prolonged ophthalmic action, upon instillation into the eye, but also could achieve delivery to the posterior segment. Without wishing to be bound by any theory, it is believed that the reversible cluster of the brimonidine loaded brimonidine loaded nano-resin particles upon instillation into the eye, declusters under shear due to eye blinking. The declustered individual drug-loaded nano-resin particles spread onto the surface of the cornea and subsequently releases the drug through ion exchange phenomenon under the effect of ions present in the ocular tissue. There occurs decrease in naso-lacrymal drainage of the drug. The overall phenomenon causes increased bioavailability of the drug along with prolonged ophthalmic action.

A further aspect of the present invention relates to said aqueous suspension as such.

Another aspect of the present invention relates to a method for the treatment of elevated intraocular pressure in human patients suffering from open angle glaucoma or ocular hypertension, comprising administering to a patient in need thereof the aqueous suspension as described above.

In a further aspect, the present invention relates to a method of reducing elevated intraocular pressure in human patients with open angle glaucoma or ocular hypertension, comprising administering brimonidine or its pharmaceutically acceptable salt, or the aqueous suspension as referred to above, wherein said method lowers the intraocular pressure in said patients by at least 2-6 mmHg; and wherein said method is effective in reducing the intraocular pressure over a time period of about 7 to 15 weeks.

In more particularly preferred embodiments, the present invention provides a method of reducing elevated intraocular pressure in humans comprising administering an aqueous suspension as referred to above that comprises reversible clusters of brimonidine loaded nano-resin particles, said clusters preferably having a D₅₀ value of at least 2 micrometers, and said brimonidine loaded nano-resin particles having a particle size distribution characterized in that the D₉₀ value is 70 nm to 900 nm, the D₅₀ value is 50 nm to 700 nm, and a suspending agent; wherein the drug is brimonidine tartrate, which is present in an amount of 0.35% w/v; the resin is polystyrene divinyl benzene sulfonate; and the suspending agent is a mixture of carbopol, polyvinylpyrrolidone and hydroxyl propyl methyl cellulose. The weight ratio between the nano-resin particles and brimonidine is about 1:1; and pH is about 7 to 8. Further, surprisingly, the aqueous suspension of the present invention despite containing a higher concentration (about 0.35% w/v) of the drug, provided an ophthalmic suspension that was very safe with no adverse effects or toxicity.

It was surprisingly found by the inventors of the present invention that the formulation of the present invention has shown statistically significant IOP lowering efficacy.

Every 1 mm Hg reduction in IOP may result in substantial prevention of visual field loss. The longer duration of effect of the present invention creates a substantial effect over a 24 hour period.

In one or more embodiments, the aqueous suspension of the present invention effectively lowers the mean intraocular pressure in humans with open angle glaucoma or ocular hypertension.

The mean decreases from baseline IOP were statistically significant within each treatment group at each follow-up time point.

In a preferred embodiment, mean IOP from baseline was decreased by at least 2-6 mmHg after once daily administration of aqueous suspension of the present invention.

In one or more embodiments, the aqueous suspension of the present invention provides an IOP reduction of at least 20% from baseline. The IOP reduction in the treated eye is either comparable to or greater than that found for the most optimized formulation of brimonidine (Alphagan® P at 0.1%).

In another embodiment, the aqueous suspension of the present invention provides an IOP reduction of about 30% to about 50% from baseline.

It is therefore very unexpected and surprising that the aqueous suspension of the present invention not only offers an improved efficacy and but also provides a significant reduction in IOP when administered once daily compared to the Alphagan P® 0.1% which was administered three times a day.

While the present invention is disclosed generally above, additional aspects are further discussed and illustrated with reference to the examples below. However, the examples are presented merely to illustrate the invention and should not be considered as limitations thereto.

### EXAMPLES

### Example 1: Aqueous Suspension of Brimonidine

The resin Amberlite® IRP69 was washed with absolute alcohol multiple times. The resin was further washed with Millipore water until a pH close to neutral (pH 7.0) was attained. The particle size distribution of the resin was measured using a Malvern Mastersizer 2000® Ver. 5.60, Malvern Instruments Ltd., Malvern, UK. The resin had a particle size distribution such that D₁₀ is 2.341 micron, D₅₀ is 5.175 micron and D₉₀ is 10.41 micron. The resin was used for preparation of the composition of Table 1 set forth below.

TABLE 1
Aqueous suspension of cationic drug: Brimonidine
Tartrate with Amberlite ® resin IRP69
of average particle size of 5 microns

| | | Amount |
|---|---|---|
| S. No. | Ingredients | (% w/v) |
| 1 | Brimonidine Tartrate | 0.35 |
| 2 | Amberlite IRP 69 | 0.35 |
| 3 | Hydroxy propyl methyl cellulose | 0.3 |
| 4 | Polyvinylpyrrolidone | 1.2 |
| 5 | Carbopol 974P (carbomer) | 0.1 |
| 6 | Benzalkonium Chloride | 0.01 |
| 7 | Edetate Disodium | 0.1 |
| 8 | N-Lauroylsarcosine sodium | 0.06 |
| 9 | Mannitol | 4.5 |
| 10 | Tromethamine q.s to adjust pH to 7.4 | 0.32 |
| 11 | Water for Injection | q.s. |

In a stainless steel (SS 316) beaker, 15% water for injection of total batch size was taken and heated to 85° C. Hydroxy propyl methyl cellulose (hypromellose 2910) was dispersed with high speed stirring to obtain uniform dispersion. The stirring was continued until the temperature reached 25° C. In another stainless steel (SS 316) beaker, a portion of water for injection of total batch size was taken to 25° C. Polyvinylpyrrolidone (povidone K-90) was dispersed in water for injection with stirring to obtain uniform dispersion.

In a stainless steel (SS 316) beaker, about 10% water for injection of total batch size was taken and heated to 65° C. Carbopol 974P was dispersed in the heated water for injection with stirring. The stirring was continued until the temperature reached 25° C. The Carbopol 974P slurry was neutralized (pH7.4) with tromethamine. The hypromellose and povidone polymer dispersions obtained above were added sequentially to the Carbopol 974P phase. The polymer mixture was autoclaved at 121° C. for 20 minutes. N-lauryl sarcosine sodium was mixed in a portion of water for injection of total batch size and added to the polymer phase after filtration through a 0.2 micron nylon filter. Mannitol was dissolved in a portion of water for injection at 50-60° C. and benzalkonium chloride, and edetate disodium were added to form a clear solution. This solution was added to the above polymer phase. 15% water for injection of total batch size was taken in a vessel and Amberlite IRP69 was dispersed with stirring. In another vessel, 15% water for injection of total batch size was taken and brimonidine tartrate was added with stirring to dissolution. This solution was filtered through 0.2 micron and 0.45 micron nylon filters. The filtered brimonidine tartrate solution was added to the above autoclaved Amberlite IRP69 dispersion and stirred for 30 minutes. The Amberlite IRP69 and brimonidine tartrate dispersion was added to the polymer mixture obtained above with stirring and stirring was continued for 1 hr. The pH was adjusted with a tromethamine solution to about 7.4. The volume of the suspension was finally made up to 100% batch size. The suspension was stirred for 60 minutes, followed by homogenization at 15000 rpm for 10 mins.

The suspension was subjected to sonication at a frequency of 33±3 KHz, for 5 seconds and a sample was withdrawn to measure the particle size distribution by a Malvern Mastersizer 2000®, Ver. 5.60, Malvern Instruments Ltd., Malvern, UK . . . . Following intervals of one minute each, the sonication process and subsequent measurement of the particle size was repeated for 5 minutes. See Table 2 set forth below for the particle size distribution.

TABLE 2
Effect of shear on the particle size distribution
of the resin particles in the suspension

| | | Volume mean diameter in microns recorded by | | | | | |
|---|---|---|---|---|---|---|---|
| | | Malvern lazer diffraction method | | | | | |
| Example No. | PSD* | Initial | 1 min | 2 min | 3 min | 4 min | 5 min |
| Comparative | D₁₀ | 1.587 | 1.380 | 1.285 | 1.351 | 1.332 | 1.348 |
| Example 1 | D₅₀ | 5.621 | 5.200 | 5.088 | 5.160 | 5.112 | 5.176 |
| | D₉₀ | 13.523 | 10.943 | 10.861 | 10.836 | 10.760 | 10.883 |

PSD*—Particle Size Distribution in Volume mean diameter in microns

Observation: It was observed that particle size distribution of the brimonidine loaded resin particles remains in the microns size, in that the D₅₀ was 5 microns at the end of 5 minutes, after application of shear by sonication at a frequency of 33±3 KHz, for 5 seconds. Further, the D₉₀ was also not affected by the shear force, and remained in the range of 10 microns.

### Preparation of Purified Nanoresin

Preparation of purified nanoresin: The resin Amberlite® IRP69 was washed multiple times with a suitable alcohol such as methanol or absolute alcohol. The resin was further washed with heated Millipore water until a pH close to neutral (pH 7.0) was attained. The washed resin was subjected to wet milling to reduce particle size to the nanometer range, having D₉₀ less than 900 nm. The washed resin and stabilized Zirconia beads were added to water for injection in a vessel containing a Teflon coated magnetic bead. The vessel was kept for wet grinding on a magnetic stirrer for about 24-48 h to obtain nano size milled resin particles. The slurry formed was passed through a 25 micron sieve to remove beads and further passed through 40 micron PP filter. The milled resin suspension obtained above was subjected to diafiltration using a 500 kD hollow fiber cartridge wherein the water extractable impurities were reduced to less than 1.0% by weight of resin. The milled resin suspension was further washed with water for injection. This slurry was lyophilized to get the dried powder form of the milled purified resin.

The particle size distribution of the milled resin was measured using a Malvern Mastersizer 2000® Ver. 5.60, Malvern Instruments Ltd., Malvern, UK. The particle size distribution was such that D₁₀=0.148 microns, D₅₀=0.24 microns and D₉₀=0.606 microns. The nanoresin was used for the preparation of aqueous suspension in Examples 2 to 5 set forth below.

### Example 2 (A) and 2 (B)

TABLE 3
Details of aqueous suspensions of the present invention

| | | Amounts- % w/v | |
|---|---|---|---|
| Ingredients | | Example | Example |
| function | Ingredients | 2(A) | 2(B) |
| Active ingredient | Brimonidine Tartrate | 0.35 | 0.15 |
| Resin | Amberlite IRP 69 | 0.35 | 0.15 |
| Polymeric vehicle | Hydroxy propyl methyl | 0.3 | 0.3 |
| | cellulose | | |
| Polymeric vehicle | Polyvinylpyrrolidone | 1.2 | 1.2 |
| Polymeric vehicle | Carbopol 974P (carbomer) | 0.1 | 0.1 |
| Preservative | Benzalkonium Chloride | 0.01 | 0.01 |
| Chelating agent | Edetate Disodium | 0.1 | 0.1 |
| Preservative | N-Lauroylsarcosine sodium | 0.06 | 0.06 |
| Osmotic agent | Mannitol | 4.5 | 4.5 |
| pH adjusting | Tromethamine q.s to | 0.32 | 0.32 |
| agent | adjust pH to 7.4 | | |
| Vehicle | Water for Injection | q.s. | q.s. |

The aqueous suspensions of Examples 2 (A) and (B) were prepared as below:

In a stainless steel (SS 316) beaker, about 15% water for injection of total batch size was taken and heated to 85° C. The specified polymeric vehicle, such as hydroxy propyl methyl cellulose (hypromellose 2910), was dispersed with high speed stirring to obtain uniform dispersion. The stirring was continued until the temperature reached 25° C. In another stainless steel (SS 316) beaker, about 12% water for injection of total batch size was taken at 25° C. Polyvinylpyrrolidone (povidone K-90) was dispersed in water for injection with stirring to obtain uniform dispersion. In the case of Example 2, in a stainless steel (SS 316) beaker, about 10% water for injection of total batch size was taken and heated to 65° C. Carbopol 974P was dispersed in the heated water for injection with stirring. The stirring was continued until the temperature reached 25° C. The Carbopol 974P slurry was neutralized (pH7.4) with tromethamine. The hypromellose and povidone polymer dispersions obtained above were added sequentially to the Carbopol 974P phase. The polymer mixture was autoclaved at 121° C. for 20 minutes. N-lauryl sarcosine sodium was mixed in a portion of water for injection and added to the polymer phase after filtration through a 0.2 micron nylon filter. Mannitol was dissolved in a portion of water for injection at 50-60° C. and benzalkonium chloride, and edetate disodium were added to form a clear solution. This solution was added to the above polymer phase. A portion of water for injection of total batch size was taken in a vessel and Amberlite IRP69 obtained as per Example 1, was dispersed with stirring. This dispersion was autoclaved at 121° C. for 20 minutes. In another vessel, a portion of water for injection was taken and brimonidine tartrate was added with stirring to dissolution. This solution was filtered through 0.2 micron and 0.45 micron nylon filters. The filtered brimonidine tartrate solution was added to the above autoclaved Amberlite IRP69 dispersion and stirred for 30 minutes.

The Amberlite IRP69 and brimonidine tartrate dispersion was added to the polymer mixture obtained above with stirring and stirring was continued for about 30 minutes to 1 hour. The volume of suspension was finally made up to 100% batch size. The suspension was stirred for about 60 minutes, followed by homogenization at 15000 rpm for 10 mins. The pH was adjusted with a tromethamine solution to about 7.4. The viscosity of the aqueous suspension of Example 2(A) was measured using a Brookfield viscometer and was found to be 19.7 cps.

### Example 3-5

The aqueous suspensions of Examples 3 to 5 were prepared in a similar manner as above but excluding steps of addition of HPMC and PVP.

TABLE 4
Details of aqueous suspensions of the present invention

| | | % w/v | | |
|---|---|---|---|---|
| Ingredients | | Example | Example | Example |
| function | Ingredients | 3 | 4 | 5 |
| Active | Brimonidine Tartrate | 0.35 | 0.35 | 0.35 |
| ingredient | | | | |
| Resin | Amberlite IRP 69 | 0.35 | 0.35 | 0.35 |
| Polymeric | Carbopol 974P | 0.1 | 0.2 | 0.3 |
| vehicle | (carbomer) | | | |
| Preservative | Benzalkonium | 0.01 | 0.01 | 0.01 |
| | Chloride | | | |
| Chelating | Edetate Disodium | 0.1 | 0.1 | 0.1 |
| agent | | | | |
| Preservative | N-Lauroylsarcosine | 0.06 | 0.06 | 0.06 |
| | sodium | | | |
| Osmotic | Mannitol | 4.5 | 4.5 | 4.5 |
| agent | | | | |
| pH adjusting | Tromethamine q.s to | 0.3 | 0.3 | 0.3 |
| agent | adjust pH to 7.4 | | | |
| Vehicle | Water for Injection | q.s. | q.s. | q.s. |

The viscosity of the aqueous suspensions of Examples 3 to 5 were measured using a Brookfield viscometer. The viscosities were found to be 8.2 cps, 12.0 cps and 97.9 cps, respectively.

### Example 6

Evaluation of chemical stability was made, for which the aqueous suspension of Example 2(A) was filled in 5 ml white opaque LDPE containers. The bottles filled with the suspension of Example 2(A) were subjected to accelerated stability conditions to determine storage stability during the shelf life of the product. The bottles were kept at different conditions. The bottles were kept in upright as well as ‘on the side’ position. The observation for assay of brimonidine is given Tables 5 and 6 below:

TABLE 5
Results of the stability data of the suspension
stored in bottles in upright position

| | | 25° C./ | 30° C./ | 40° C./ |
|---|---|---|---|---|
| | | 40% RH | 35% RH | 25% RH |
| Assay | Initial | 6 M | 6 M | 6 M |
| % Brimonidine | 101.1 | 99.58 | 96.11 | 96.91 |
| tartrate | | | | |

TABLE 6
Results of the stability data of the suspension stored
in bottles & kept ‘on the side’ position

| | | 25° C./ | 30° C./ | 40° C./ |
|---|---|---|---|---|
| | | 40% RH | 35% RH | 25% RH |
| Assay | Initial | 6 M | 6 M | 6 M |
| % brimonidine | 101.1 | 99.27 | 98.01 | 95.74 |
| tartrate | | | | |

The results in Tables 5 6 indicate that there was no significant change in assay of brimonidine after long term storage. The assay of the drug remained within specified limits and no degradation products or impurities were observed upon storage. Also, there was no significant increase in related substances. The suspension according to the present invention remained chemically stable during the shelf life of the product. The suspension is room temperature stable.

### Example 7

This example describes the effect of shear on the reversible clusters of brimonidine loaded nano-resin particles suspended in Example 2(A), which decluster into individual drug-loaded nano-resin particles when subjected to shear, such as a shear resulting from blinking in the eye. This effect was measured in terms of particle size distribution, initially and upon application of shear.

Procedure: The test samples were subjected to shear by placing the vials containing the suspension on a bath sonicator (Model type: MC-109 and SI no-1909; Mfg. by Oscar Ultrasonic Pvt. Ltd.) and shear was applied in the form of a sonication frequency of 33±3 kHz for 5 seconds after which the sample was withdrawn to measure the particle size distribution. Following intervals of 1 minute each the process was repeated 5 times.

The particle size measurement was done using a Malvern Mastersizer 2000®, Ver. 5.60, Malvern Instruments Ltd., Malvern, UK, but the analyser's sonication means were not used. The sample was only subjected to mild stirring by a mechanical stirrer. The observations are summarized below in Table 7.

TABLE 7
Effect of shear on the particle size
distribution of the resin particles:

| | | Volume mean diameter in microns recorded by | | | | | |
|---|---|---|---|---|---|---|---|
| | | Malvern lazer diffraction method | | | | | |
| Example No. | PSD* | Initial | 1 min | 2 min | 3 min | 4 min | 5 min |
| Example2(A) | D₁₀ | 0.852 | 0.475 | 0.338 | 0.151 | 0.145 | 0.140 |
| | D₅₀ | 19.549 | 13.882 | 0.996 | 0.254 | 0.229 | 0.213 |
| | D₉₀ | 58.970 | 49.111 | 30.405 | 1.398 | 0.512 | 0.449 |

PSD*—Particle Size Distribution in Volume mean diameter in microns

Observations: It was found that clusters of brimonidine loaded nano-resin particles of Example 2(A), disintegrated completely as shear was applied to the suspension. This was evident by the decrease in the particle size observed upon application of shear/sonication as shown in Table 7. The D₅₀ of drug-resin nanoparticles was initially about 19.5 microns, which upon application of shear at regular interval for 5 minutes disintegrated and converted into individual drug-resin nanoparticles having D₅₀ of 0.213 micron (213 nm).

On the contrary, in case of Comparative Example 1 there was no disintegration and the particle size did not change. This is apparent from Table 2. As shown in Table 2, the D₅₀ of the suspended drug-resin particles was initially about 5.2 microns and even after application of shear at regular interval for 5 minutes, the size of the particles did not changed and remained almost the same, viz. 5.176 microns.

### Example 8

A Randomized, Multi-Center, Investigator-Masked, Parallel Group, Equivalence Study of Once Daily Brimonidine Tartrate Ophthalmic Suspension Compared with Three Times Daily Alphagan® P in Humans with Open Angle Glaucoma or Ocular

### Hypertension

This study was designed to obtain estimates on the mean IOP at each of the 3 time points (8:00 AM, 10:00 AM, 4:00 PM) on week 2, week 6 and week 12 in order to compare brimonidine tartrate 0.35% ophthalmic suspension, dosed QD at 8:00 AM and brimonidine tartrate 0.1%, dosed TID at approximately 8:00 AM, 2:00 PM, and 8:00 PM.

The primary objective was to evaluate the efficacy of once daily (QD) dosing with brimonidine tartrate ophthalmic suspension 0.35% as prepared according to the Example 2(A) above compared with a brimonidine tartrate ophthalmic solution 0.1% (“Alphagan”) dosed TID in human subjects (i.e. patients) with open-angle glaucoma, or ocular hypertension.

The secondary objective was to evaluate the safety of QD dosing with a brimonidine tartrate ophthalmic suspension 0.35% of the present invention compared with a brimonidine tartrate ophthalmic solution 0.1% dosed TID in subjects with open-angle glaucoma, or ocular hypertension.

Subjects who were chronically treated with ocular hypotensive medications were required to undergo appropriate washout periods prior to study entry to minimize any residual effects of other active ocular hypotensive medications.

Qualified humans with open angle glaucoma, pseudoexfoliation, pigment dispersion, or ocular hypertension were stratified into 2 groups based on the Visit 2/8:00 AM IOP in study eye: IOP≤25 mm Hg or IOP>25 mm Hg. Within each stratum and site, subjects were randomly assigned (1:1 ratio) at Visit 2. Approximately 666 subjects were enrolled.

Subjects having open angle glaucoma, (with or without pseudo exfoliation, pigment dispersion component) or ocular hypertension in both eyes and likely to be controlled on monotherapy were included.

The primary efficacy variable was, the time-matched mean IOP (study eye) at each of the 3 time points (8:00 AM, 10:00 AM, 4:00 PM) on week 2, week 6 and week 12. These sample values were used to compute the point estimates for the primary estimand defined by the difference across treatment groups in IOP at each of the 9 time points (3 times within each of 3 days).

The Secondary Efficacy Variables were:

The time-matched change from baseline mean IOP (study eye) at each of the 3 time points (8:00 AM, 10:00 AM, 4:00 PM) on week 2, week 6 and week 12.

Standard deviation IOP (study eye) at each of the 3 time points (8:00 AM, 10:00 AM, 4:00 PM) on week 2, week 6 and week 12 and;

Time-matched percent change from baseline in IOP (study eye) at each of the 3 time points (8:00 AM, 10:00 AM, 4:00 PM) on week 2, week 6 and week 12.

The primary efficacy analyses was completed by constructing two-sided 95% confidence intervals on the difference in time-matched IOP measurements between the two treatment arms at each time point (8 am, 10 am, and 4 pm) for week 2, week 6 and week 12 (a total of 9 time points) using the per protocol population. These confidence intervals were constructed using a two-sample t-distribution with pooled variance. In the event that the sample variances were significantly different then the confidence intervals used were constructed via the Satterthwaite adjustment. The p-values for testing equivalence of these time-matched means between treatment groups were generated using the TOST (two one-sided tests) procedure at each of the equivalence limits (−1.0, 1.0) and (−1.5, 1.5).

Each of the secondary efficacy endpoints were summarized using descriptive statistics by treatment group and time point using the PP population. The 95% confidence intervals at each time point within treatment groups were displayed along with the 95% confidence intervals on the time matched differences between treatment groups for each of these secondary endpoints.

The primary safety analysis summarized ocular (in either eye) and non-ocular TEAEs for all treated subjects using discrete summaries at the subject level by system organ class and preferred term for each treatment group. A TEAE is defined as occurring after the first dose of study medication.

Slit lamp biomicroscopy was performed at every visit during the study to observe the overall health of the eye, including the lid/lashes, conjunctiva, cornea anterior chamber, iris, and lens.

### Visits

Visit 1: (Day−42 to Day−1): Screening
Visit 2: (Day 0) Baseline/Randomization
Visit 3: (Week 2±2 Days) Efficacy/Safety Evaluation
Visit 4: (Week 6±2 Days) Efficacy/Safety Evaluation
Visit 5: (Week 12±2 Days) Efficacy/Safety Evaluation and study exit

During said visits various adverse events were checked, such as somnolence, ocular hyperemia, nervous system disorders, oral dryness (all visits), and dilated ophthalmoscopy (Visits 1, and Visit 5): items to be observed were retina, macula, choroids, optic nerve, and optic disc pallor examination measures were summarized at each visit using discrete summary statistics.

Visual acuity: Data was summarized at each visit, using continuous and discrete summaries, including change from baseline in the number of lines and the proportion of subjects with a worsening of ≥3 lines from baseline.

Heart rate and blood pressure: Data was summarized using continuous summary statistics (mean, standard deviation, minimum, median, and maximum) by visit including change from baseline summaries.

Results are set forth in Tables 8 and 9 set forth below.

TABLE 8
Subject Disposition (ITT population)

| | Brimonidine | | Alphagan | | Total | |
|---|---|---|---|---|---|---|
| Subject | n(%) | | n(%) | | n(%) | |
| Disposition | (N = 341) | | (N = 341) | | (N = 682) | |
| Subjects | | | | | 945 | |
| Screened | | | | | | |
| Screen Failure | | | | | 263 | |
| Randomized | 341 | (100.0) | 341 | (100.0) | 682 | (100.0) |
| Randomized but | 4 | (1.2) | 0 | (0) | 4 | (0.6) |
| not dosed | | | | | | |
| Safety | 337 | (98.8) | 341 | (100.0) | 678 | (99.4) |
| Population | | | | | | |
| ITT Population | 341 | (100.0) | 341 | (100.0) | 682 | (100.0) |
| PP Population | 258 | (75.7) | 265 | (77.7) | 523 | (76.7) |
| Completed | 293 | (85.9) | 307 | (90.0) | 600 | (88.0) |
| Study | | | | | | |
| Discontinued | 48 | (14.1) | 34 | (9.9) | 82 | (12.0) |
| from study | | | | | | |

ITT population: All subjects who are randomized into the study;

Per Protocol Population: The PP population includes all randomized subjects who meet all inclusion/exclusion criteria, do not miss the scheduled applications for more than 3 consecutive days, and complete evaluations at week 2, week 6, and week 12 with no protocol violations that would affect the treatment evaluation and their compliance is between ≥75% to ≤125%

TABLE 9
Difference in Mean IOP (GoldmannTonometer)
between Treatments (PP Population)
Study EYE:

| | | Brimonidine | Alphagan |
|---|---|---|---|
| | | N = 258 n(%) | N = 265 n(%) |
| Visit | Time Point | N, Mean (SD) | N, Mean (SD) |
| 2 | 8AM +/− 30 m | 258, 24.76 (2.390) | 265, 25.21 (2.397) |
| | 10AM +/− 30 m | 258, 23.32 (3.134) | 265, 23.42 (3.109) |
| | 4PM +/− 30 m | 258, 21.94 (3.590) | 265, 22.32 (3.364) |
| 3 | 8AM +/− 30 m | 258, 20.03 (3.567) | 265, 20.45 (3.401) |
| | 10AM +/− 30 m | 258, 16.86 (3.442) | 265, 17.87 (3.177) |
| | 4PM +/− 30 m | 258, 17.94 (3.457) | 265, 17.49 (3.085) |
| 4 | 8AM +/− 30 m | 258, 20.03 (3.888) | 265, 20.55 (3.504) |
| | 10AM +/− 30 m | 258, 17.00 (3.682) | 265, 17.98 (3.399) |
| | 4PM +/− 30 m | 258, 17.53 (3.160) | 265, 17.35 (3.237) |
| 5 | 8AM +/− 30 m | 257, 20.35 (3.746) | 263, 20.79 (3.724) |
| | 10AM +/− 30 m | 256, 17.38 (3.401) | 262, 18.31 (3.475) |
| | 4PM +/− 30 m | 254, 17.99 (3.309) | 263, 17.57 (3.579) |

For the testing of equivalence of brimonidine (i.e. the aqueous suspension according to the present invention) and Alphagan on the difference in time-matched IOP measurements between the two treatment arms at each time point (8 am, 10 am, and 4 pm) for week 2, 6 and 12 the results are significant. Hence, the primary objective of the study was met, i.e. the brimonidine formulation according to the present invention is equivalent to Alphagan.

With respect to adverse events, it was already observed in a study with 70 patients receiving the aqueous suspension of the present invention that particularly ocular hyperemia, somnolence and nervous system disorders occurred. In said study the incidence of side effects was as follows:

| | |
|---|---|
| Ocular hyperaemia: | 32.9% of the patients |
| Gastrointestinal disorders | 22.9% of the patients |
| Dry Mouth | 22.9% of the patients |
| Nervous system disorders | 20.0% of the patients |
| Somnolence | 18.6% of the patients |

1. An aqueous suspension comprising: (a) reversible clusters of brimonidine loaded nano-resin particles and (b) a suspending agent, wherein said brimonidine loaded nano-resin particles have a particle size distribution wherein the D₉₀ value is between 70 to 900 nm and D₅₀ value is between 50-700 nm, and wherein said suspension is for use in the treatment of elevated intraocular pressure in human patients suffering from open angle glaucoma or ocular hypertension.

2. The aqueous suspension according to claim 1, wherein the D₉₀ value is 200 nm to 700 nm.

3. The aqueous suspension according to claim 1, wherein the D₅₀ value of the nano-resin particles is 50 to 700 nm.

4. The aqueous suspension according to claim 3, wherein the D₅₀ value of the nano-resin particles is 100 to 500 nm.

5. The aqueous suspension according to claim 3, wherein the D₅₀ value of the nano-resin particles is between 150 to 350 nm.

6. The aqueous suspension according to claim 3, wherein the D₅₀ value of the nano-resin particles is between 200 to 300 nm

7. The aqueous suspension according to claim 1, wherein the reversible clusters comprise brimonidine as a pharmaceutically acceptable salt or solvate.

8. The aqueous suspension according to claim 7, wherein the brimonidine is brimonidine tartrate.

9. The aqueous suspension according to claim 1, wherein said suspension is administered into the eye of the patients once daily.

10. The aqueous suspension for use according to claim 1, wherein said treatment further comprises the steps of:
(a) identifying the eye disease history of the patient to be treated before commencing the treatment with the aqueous suspension;
(b) having ophthalmic examinations performed within at least 16 weeks, preferably within at least 12 weeks, after commencing the treatment with the aqueous suspension, said examinations comprising checking and/or monitoring the appearance of somnolence, ocular hyperemia, nervous system disorders or oral dryness;
(c) having optional additional ophthalmologic examinations performed based on patient symptoms at intervals determined by an ophthalmologist.

11. The aqueous suspension according to claim 1, wherein the mean intraocular pressure is lowered by at least 2-6 mm Hg.

12. The aqueous suspension according to claim 1, wherein said suspension comprises about 0.05% to about 0.5% weight by volume brimonidine or a pharmaceutically acceptable salt thereof.

13. The aqueous suspension according to claim 1, wherein said suspension comprises about 0.2% to about 0.5% weight by volume brimonidine or a pharmaceutically acceptable salt thereof.

14. The aqueous suspension according to claim 1, wherein said suspension comprises about 0.35% weight by volume brimonidine tartrate.

15. A method for the treatment of elevated intraocular pressure in a human patient suffering from open angle glaucoma or ocular hypertension, comprising administering to a patient in need thereof an aqueous suspension according to claim 1.

16. A method of reducing elevated intraocular pressure in a human patient with open angle glaucoma or ocular hypertension, comprising administering brimonidine or its pharmaceutically acceptable salt, wherein said method lowers the intraocular pressure in said patient by at least 2-6 mmHg; and said method is effective in reducing the intraocular pressure over a time period of about 7 to 15 weeks.

17. A method of reducing elevated intraocular pressure in a human patient with open angle glaucoma or ocular hypertension, comprising administering an aqueous suspension according to claim 1, wherein said method lowers the intraocular pressure in said patient by at least 2-6 mmHg; and said method is effective in reducing the intraocular pressure over a time period of about 7 to 15 weeks.

18. The method according to claim 15, wherein brimonidine or its pharmaceutically acceptable salt is present at a concentration of about 0.05% to 0.5% weight by volume.

19. The method according to claim 15, wherein brimonidine or its pharmaceutically acceptable salt is brimonidine tartrate and is present at a concentration of about 0.35% weight by volume.

20. The method according to claim 15, wherein said method is effective in reducing the intraocular pressure for at least 12 weeks.

21. The method according to claim 15, wherein brimonidine is administered into the eye of the patient once daily.

22. A method of reducing elevated intraocular pressure in a human patient with open angle glaucoma or ocular hypertension, comprising administering brimonidine or its pharmaceutically acceptable salt, wherein after administration the patient achieved >20% intraocular pressure reduction from baseline which was sustained for at least 12 weeks.

23. A method of reducing elevated intraocular pressure in a human patient with open angle glaucoma or ocular hypertension, comprising administering an aqueous suspension according to claim 1, wherein after administration the patient achieved >20% intraocular pressure reduction from baseline which was sustained for at least 12 weeks.

24. The method according to claim 22, wherein the patient achieved >40% intraocular pressure reduction from baseline which was sustained for at least 12 weeks.

25. The method according to claim 22, wherein brimonidine or its pharmaceutically acceptable salt is present at a concentration of about 0.05% to 0.5% weight by volume.

26. The method according to claim 22, wherein brimonidine or its pharmaceutically acceptable salt is brimonidine tartrate and is present at a concentration of about 0.35% weight by volume.

27. The method according to claim 22, wherein brimonidine is administered into the eye of the patients once daily.
